(19) United States
(12) Patent Application Publication
(10) Pub. No.: US 2003/0202973 A1
(43) Pub. Date: Oct. 30, 2003

(54) Treatment of refractory human tumors with epidermal growth factor receptor and HER1 mitogenic ligand (EGFRML) antagonists

(75) Inventor: George Pieczenik, New York, NY (US)

Correspondence Address: DR. GEORGE PIECZENIK, APT. 1F, 412 EAST 55TH STREET, NEW YORK, NY, 10022, US

(73) Assignee: Dr. George Pieczenik, New York, NY (US)

(21) Appl. No.: 10/063,930

(22) Filed: May 27, 2002

Related U.S. Application Data
(60) Provisional application No. 60/319,212, filed on Apr. 29, 2002.
(60) Provisional application No. 60/319,269, filed on May 26, 2002.

(51) Int. Cl. A61K 39/395; A61N 5/00

(52) U.S. Cl. 424/141.1; 600/1

(57) ABSTRACT

A method of inhibiting the growth of refractory tumors that are stimulated by mitogenic ligands of epidermal growth factor receptor in human patients, comprising treating the human patients with an effective amount of a mitogenic ligand antagonist.

### CROSS REFERENCE TO RELATED APPLICATIONS

claim priority to U.S. Provisional Application 60319212 Filed Apr. 29, 2002 and U.S. Provisional Application 60319269 Filed May 26, 2002

### Federal Research Statement

[No Federal Research funds were used for this invention]

### BACKGROUND OF INVENTION

In the United States, cancer is the second leading cause of death after heart attacks. Progress in new therapy development depends on understanding the mechanisms of cell proliferation in both normal cells and cancerous cells.

Normal cells proliferate by the highly controlled activation of growth factor receptors by their respective ligands. Examples of such receptors are the growth factor receptor tyrosine kinases.

Cancer cells also proliferate by the activation of growth factor receptors by mitogenic ligands, but lose the careful control of normal proliferation. The loss of control may be caused by numerous factors, such as the overexpression of growth factors and/or receptors, and autonomous activation of biochemical pathways regulated by mitogenic growth factors.

Some examples of receptors involved in tumorigenesis are the receptors for epidermal growth factor (EGFR), platelet-derived growth factor (PDGFR), insulin-like growth factor (IGFR), nerve growth factor (NGFR), and fibroblast growth factor (FGF).

Some examples of mitogenic ligands that bind these receptors that are involved in tumorigenesis are epidermal growth factor (EGF), nerve growth factor (NGF), and fibroblast growth factor (FGF).

Members of the epidermal growth factor (EGF) receptor family are particularly important growth factor receptor tyrosine kinases associated with tumorigenesis of epidermal cells. The first member of the EGF receptor family to be discovered was the glycoprotein having an apparent molecular weight of approximately 165 kD. This glycoprotein, which was described by Mendelsohn et al. in U.S. Pat. No. 4,943,533, is known as the EGF receptor (EGFR) and also as human EGF receptor-1 (HER1). The EGFR is overexpressed on many types of epidermoid tumor cells.

Epidermal growth factor (EGF) and transforming growth factor alpha (TGF-alpha) are two known well-known ligands of EGF receptor (EGFR). Naturally occurring ligands, inter alia, which bind EGFR are to be called epidermal growth factor receptor mitogenic ligands and abbreviated as EGFRML.

The inhibition of EGFRMLs binding EGFR is the logic behind this invention. It is the key to the methods below described for treating and palliating the course and progression of refractory cancers. Inhibiting naturally occurring mitogens will inhibit the growth of tumors.

Examples of tumors that express EGF receptors include glioblastomas, as well as cancers of the lung, breast, head and neck, and bladder. The amplification and/or overexpression of the EGF receptors on the membranes of tumor cells are associated with a poor prognosis. Poor prognosis may also be a consequence of an excess the mitogenic epidermal growth factor stimulating the EGF receptors.

Treatments of cancer traditionally include chemotherapy or radiation therapy. Some examples of chemotherapeutic agents include doxorubicin, cisplatin, and taxol. The radiation can be either from an external beam or from a source placed inside a patient, i.e., brachytherapy.

Another type of treatment includes antagonists of growth factors or growth factor receptors involved in the proliferation of cells. Such antagonists neutralize the activity of the growth factor and/or receptor, and inhibit the growth of tumors that express the receptor.

For example, U.S. Pat. No. 4,943,533 describes a murine monoclonal antibody called 225 that binds to the EGF receptor. The 225 antibody is able to inhibit the growth of cultured EGFR-expressing tumor lines as well as the growth of these tumors in vivo when grown as xenografts in nude mice. See Masui et al., Cancer Res. 44, 5592-5598(1986).

A disadvantage of using murine monoclonal antibodies in human therapy is the possibility of a human anti-mouse antibody (HAMA) response due to the presence of mouse antibody sequences. This disadvantage can be minimized, but not eliminated, by replacing the entire constant region of a murine (or other non-human mammalian) antibody with that of a human constant region. Replacement of the constant regions of a murine antibody with human sequences is usually referred to as humanized or chimerization.

The humanization process can be made even more effective by also replacing the framework variable regions of a murine antibody with the corresponding human sequences. The framework variable regions are the variable regions of an antibody other than the hypervariable regions. The hypervariable regions are also known as the complementarity-determining regions (CDRs).

The replacement of the constant regions and framework variable regions with human sequences is usually referred to as humanization. The humanized antibody is less immunogenic (i.e. elicits less of a HAMA response) as more murine sequences are replaced by human sequences. Unfortunately, both the cost and effort increase as more regions of a murine antibodies are replaced by human sequences. It should be noted that human sequences are inferred from the homology among human immunoglobulin sequences versus the homology among murine immunoglobin sequences. The real nature of the immunological recognition of human sequences versus mouse sequences in humans has still not been solved. The use of the term humanized is a semantic term based on sequence analysis as opposed to a functional assay for the immunological nature of being human.

Larger size proteins induce an immunological response. Small peptides under 12 amino acids generally do not. Therefore, another approach to reducing the immunogenicity of antibodies is the use of small fragments of antibodies. Aboud-Pirak et al., Journal of the National Cancer Institute 80, 1605-1611 (1988), found that the antibody and its bivalent F(ab′).sub.2 fragment both retarded tumor growth in vivo, although the fragment was less efficient. Interestingly, he found that the monovalent Fab fragment of the antibody still binds the EGF receptor but did not retard tumor growth. This suggests that just binding to an epitope of EGFR is not sufficient to retard tumor growth. Binding toe EGF receptor must be accompanied by another as yet undetermined function to inhibit tumor growth. Perhaps, phosphorylation of a tyrosine kinase is such a function.

Therefore, this experiment suggests that blocking EGFRMLs from binding EGF receptors may be more effective in inhibiting tumor growth than just directly binding to EGF receptors.

EGFRML's may induce a change in receptor function that simple binding to the receptor will not. EGFRML's may displace endogenous peptide found in EGF receptors or displace an interaction within the EGF receptor protein complex. Therefore, arresting or blocking the EGFRML from displacing any functional aspects of the EGF receptor may retard tumor growth.

Combinations of some of the techniques mentioned above have been attempted. Baselga et al. reported anti-tumor effects of the chemotherapeutic agent doxorubicin with anti-EGFR monoclonal antibodies in the Journal of the National Cancer Institute 85,1327-1333 (1993).

Bonnen, U.S. Pat. No. 4,846,782 reported a combination of radiation with an adjuvant such as interferon had some success.

However, none of the above have been directed specifically at treating tumors refractory to conventional chemotherapy and radiation. Refractory tumors lead to rapid disease progression, usually with a poor prognosis. Currently there is little that can be done for patients with tumors refractory to conventional cancer treatment.

Based on the foregoing, there is a need for an improved method of treating refractory tumors in humans.

### SUMMARY OF INVENTION

This invention provides a method of inhibiting the growth of refractory tumors that are stimulated by a mitogenic ligand of epidermal growth factor receptor (EGFR) in human patients. The method comprises treating the human patients with an effective amount of a mitogenic ligand antagonist, EGFRML, rather than an EGFR antagonist.

Mitogenic ligand antagonist, EGFRML, include antagonist to epidermal growth factor (EGF) and transforming growth factor-alpha (TGF-alpha). EGF and TFG-alpha both bind EGF receptor and act as mitogens.

In another embodiment, the method of the present invention comprises treating human patients with a combination of an effective amount of a mitogenic ligand antagonist and a chemotherapeutic agent.

In another embodiment, the method of the present invention comprises treating human patients with a combination of an effective amount of a mitogenic ligand antagonist and radiation.

### DETAILED DESCRIPTION

The present invention provides an improved method for treating refractory tumors in humans.

Refractory Tumors Refractory tumors include tumors that fail or are resistant to treatment with chemotherapeutic agents alone, radiation alone or combinations thereof. For the purposes of this specification, refractory tumors also encompass tumors that appear to be inhibited by treatment with chemotherapeutic agents and/or radiation but recur up to five years, sometimes up to ten years or longer after treatment is discontinued.

The types of refractory tumors that can be treated in accordance with the invention are any refractory tumors that are stimulated by a mitogenic ligand of EGF receptors (EGFRML). Examples of mitogenic ligands that stimulate EGFR include EGF and TGF-alpha, inter alia.

The EGFR family of receptors includes EGFR, which is also referred to in the literature as HER1. In this specification, EGFR refers to the specific member of the EGFR family of receptors called EGFR/HER1 (EGFR).

The refractory tumors treatable by the present invention are endogenous tumors native to human patients. These tumors are more difficult to treat than exogenous human tumor xenografts that were treated in animals. See, for example, Prewett et al., Journal of Immunotherapy 19, 419-427 (1997).

Some examples of refractory tumors include carcinomas, gliomas, sarcomas, adenocarcinomas, adenosarcomas and adenomas. Such tumors occur in virtually all parts of the human body, including every organ. The tumors may, for example, be present in the breast, heart, lung, small intestine, colon, spleen, kidney, bladder, head and neck, ovary, prostate, brain, pancreas, skin, bone, bone marrow, blood, thymus, uterus, testicles, cervix, and liver.

EGFR/HER1 Mitogenic Ligand (EGFRML) Antagonists The refractory tumors of the present invention can be treated with a ligand antagonist. For the purposes of this specification, a ligand antagonist is any substance that inhibits the stimulation of EGFR/HER1 by a mitogenic ligand. Such inhibition of stimulation inhibits the growth of cells that express EGFR/HER1.

The growth of refractory tumors is sufficiently inhibited in the patient to prevent or reduce the progression of the cancer (i.e. growth, invasiveness, metastasis, and/or recurrence). The EGFRML antagonists of the present invention can be cytostatic or inhibit the growth of the refractory tumor.

No particular mechanism of inhibition is implied as operating in the present invention. Nevertheless, EGFR tyrosine kinases are generally activated by means of phosphorylation events. Accordingly, phosphorylation assays are useful in predicting the antagonists useful in the present invention. Some useful assays for EGFR tyrosine kinase activity are described in Panek et al., Journal of Pharmacology and Experimental Therapeutics 283, 1433-1444 (I 997) and in Batley et al., Life Sciences 62, 143-150 (1998). The description of these assays is incorporated herein by reference.

EGFR/HER1 mitogenic ligand antagonists (EGFRML) include biological molecules or small molecules. Biological molecules include all lipids and polymers of monosaccharides, amino acids and nucleotides having a molecular weight greater than 600. Thus, biological molecules include, for example, oligosaccharides and polysaccharides; oligopeptides, polypeptides, peptides, and proteins; and oligonucleotides and polynucleotides. Oligonucleotides and polynucleotides include, for example, DNA and RNA.

Biological molecules further include derivatives of any of the molecules described above. For example, derivatives of biological molecules include lipid and glycosylation derivatives of oligopeptides, polypeptides, peptides and proteins. Derivatives of biological molecules further include lipid derivatives of oligosaccharides and polysaccharides, e.g. lipopolysaccharides. Most typically, biological molecules are antibodies, or functional equivalents of antibodies.

Functional equivalents of antibodies have binding characteristics comparable to those of antibodies, and inhibit the growth of cells that express EGFR. Such functional equivalents include, for example, chimerized, humanized and single chain antibodies as well as fragments thereof and peptide mimics of antibody bindings to ligands, as well as peptide mimetics of antibody bindings to ligand. These shall be called antibody mimetics (AbMimes).

Functional equivalents of antibodies (AbMimes) also include polypeptides with amino acid sequences substantially the same as the amino acid sequence of the variable or hypervariable regions of the antibodies of the invention. An amino acid sequence that is substantially the same as another sequence, but that differs from the other sequence by means of one or more substitutions, additions, and/or deletions, is considered to be an equivalent sequence. Preferably, less than 50%, more preferably less than 25%, and still more preferably less than 10%, of the number of amino acid residues in a sequence are substituted for, added to, or deleted from the protein.

The functional equivalent of an antibody can be a chimerized or humanized antibody. A chimerized antibody comprises the variable region of a non-human antibody and the constant region of a human antibody. A humanized antibody comprises the hypervariable region (CDRS) of a non-human antibody. The variable region other than the hypervariable region, e.g. the framework variable region, and the constant region of a humanized antibody are those of a human antibody.

For the purposes of this application, suitable variable and hypervariable regions of non-human antibodies may be derived from antibodies produced by any non-human mammal in which monoclonal antibodies are made. Suitable examples of mammals other than humans include, for example, rabbits, rats, mice, horses, goats, or primates. Mice are preferred.

Functional equivalents further include fragments of antibodies that have binding characteristics that are the same as, or are comparable to, those of the whole antibody. Suitable fragments of the antibody include any fragment that comprises a sufficient portion of the hypervariable (i.e. complementarity determining) region to bind specifically, and with sufficient affinity, to ligands which bind EGF receptor to inhibit growth of cells that express such receptors. Such fragments may, for example, contain one or both Fab fragments or the F(ab′).sub.2 fragment. Preferably the antibody fragments contain all six complementarity determining regions of the whole antibody, although functional fragments containing fewer than all of such regions, such as three, four or five CDRs, are also included.

The preferred fragments are single chain antibodies, or Fv fragments. Single chain antibodies are polypeptides that comprise at least the variable region of the heavy chain of the antibody linked to the variable region of the light chain, with or without an interconnecting linker. Thus, Fv fragment comprises the entire antibody combining site. These chains may be produced in bacteria, in eukaryotic cells or on bacteriophage. These chains can be expressed on the surface of the vectors so as to be accessible to binding as described in Pieczenik U.S. Pat. No. 5,866,363, herein, incorporated by reference.

The antibodies and functional equivalents may be members of any class of immunoglobulins, such as: IgG, IgM, IgA, IgD, or IgE, and the subclasses thereof. The preferred antibodies are members of the IgG1 subclass. The functional equivalents may also be equivalents of combinations of any of the above classes and subclasses.

Antibodies may be made from the desired receptor as an immunogen by methods that are well known in the art. The receptors are either commercially available, or can be isolated by well known methods. For example, methods for isolating and purifying EGFR are found in Spada, U.S. Pat. No. 5,646,153.The patent is incorporated herein by reference.

Methods for making monoclonal antibodies include the immunological method described by Kohler and Milstein in Nature 256, 495-497 (1975) and by Campbell in “Monoclonal Antibody Technology, The Production and Characterization of Rodent and Human Hybridomas” in Burdon et al., Eds, Laboratory Techniques in Biochemistry and Molecular Biology, Volume 13, Elsevier Science Publishers, Amsterdam (1985). The recombinant DNA method is described by Huse et al. in Science 246, 1275-1281 (1989) and Pieczenik in U.S. Pat. No. 5,866,363 filed in 1985, issued 1999 and incorporated by reference.

Briefly, in order to produce monoclonal antibodies, a host mammal is inoculated with a receptor ligand or a fragment of a receptor ligand, as described above, and then, optionally, boosted. In order to be useful, the receptor fragment must contain sufficient amino acid residues to define the epitope of the molecule being detected. If the fragment is too short to be immunogenic, it may be conjugated to a carrier molecule. Some suitable carrier molecules include keyhole limpet hemocyanin and bovine serum albumin. Conjugation may be carried out by methods known in the art. One such method is to combine a cysteine residue of the fragment with a cysteine residue on the carrier molecule.

Spleens are collected from the inoculated mammals a few days after the final boost. Cell suspensions from the spleens are fused with a tumor cell. The resulting hybridoma cells that express the antibodies are isolated, grown, maintained in culture and selected for desired binding.

Alternatively, spleens can be collected from the naive uninnoculated mammals as described in Pieczenik U.S. Pat. No. 5,866,363 and incorporated by reference. Cell suspensions from the spleens are fused with a tumor cell. The resulting hybridoma cells that express the antibodies are isolated, grown, maintained in culture and selected for desired binding.

Suitable monoclonal antibodies as well as mitogenic growth factor ligands for making them are also available from commercial sources, for example, from Upstate Biotechnology, Santa Cruz Biotechnology of Santa Cruz, Calif., Transduction Laboratories of Lexington, Ky., R&D Systems Inc of Minneapolis, Minn., and Dako Corporation of Carpinteria, Calif.

Methods for making chimeric and humanized antibodies are also known in the art.

For example, methods for making chimeric antibodies include those described in U.S. patents by Boss (Celltech) and by Cabilly (Genentech). See U.S. Pat. Nos. 4,816,397 and 4,816,567, respectively. Methods for making humanized antibodies are described, for example, in Winter, U.S. Pat. No. 5,225,539.

The preferred method for the humanization of antibodies is called CDR-grafting. In CDR-grafting, the regions of the mouse antibody that are directly involved in binding to antigen, the complementarity determining region or CDRs, are grafted into human variable regions to create “reshaped human” variable regions. These fully humanized variable regions are then joined to human constant regions to create complete “fully humanized” antibodies. These are semantic definitions based on sequence analysis not functional definitions based on an assay for “humanized”. Such a functional assay does not exist at present.

In order to create fully humanized antibodies that bind well to an antigen, it is advantageous to design the reshaped human variable regions carefully. The human variable regions into which the CDRs will be grafted should be carefully selected, and it is usually necessary to make a few amino acid changes at critical positions within the framework regions (FRs) of the human variable regions.

For example, the reshaped human variable regions may include up to ten amino acid changes in the FRs of the selected human light chain variable region, and as many as twelve amino acid changes in the FRs of the selected human heavy chain variable region. The DNA sequences coding for these reshaped human heavy and light chain variable region genes are joined to DNA sequences coding for the human heavy and light chain constant region genes, preferably .gamma.1 and kappa., respectively. The reshaped humanized antibody is then expressed in mammalian cells and its affinity for its target compared with that of the corresponding murine antibody and chimeric antibody.

Methods for selecting the residues of the humanized antibody to be substituted and for making the substitutions are well known in the art. See, for example, Co et al., Nature 351, 501-502 (1992); Queen et al., Proc. Natl. Acad. Sci. 86, 10029-1003 (1989) and Rodrigues et al., Int. J. Cancer, Supplement 7, 45-50 (1992). A general method for humanizing monoclonal antibodies is described by Goldstein et al. in PCT application WO 96/40210. This method can be adapted to humanizing and reshaping antibodies to EGFRML's as well as against growth factor receptor tyrosine kinases as described.

Methods for making single chain antibodies are also known in the art. Some suitable examples include those described by Wels et al. in European patent application 502 812.

Other methods for producing the functional equivalents described above are disclosed in U.S. Pat. No. 5,658,570 and U.S. Pat. No. 5,693,780.

Preferred embodiments are anti-EGFR mitogenic ligand antibodies are the chimerized, humanized, and single chain antibodies derived from a murine antibody to be defined as an anti-EGFRML antibody.

This antibody should be able to inhibit the growth of cultured EGFR/HER1-expressing tumor cells in vitro as well as in vivo when grown as xenografts in nude mice. See Masui et al., Cancer Res. 44, 5592-5598 (1986). A treatment regimen combining anti-EGFR-ligand mAb plus doxorubicin or cisplatin should exhibit therapeutic synergy against several well established human xenograft models in mice. Basalga et al., J. Natl. Cancer Inst. 85, 1327-1333 (1993). In one embodiment of the present invention, human patients with refractory head and neck squamous cell carcinoma can be treated with a combination of an anti-EGFRML antibody, inter alia and cisplatin. These patients will have failed prior treatment with radiation alone, chemotherapy alone or combinations thereof. The anti-EGFRML antibody antagonist should inhibit the growth of refractory tumors.

The chimerized, humanized, and single chain antibodies are derived from murine anti-EGFRML antibody. Alternatively, the various fragments needed to prepare the chimerized, humanized, and single chain anti-EGFR-ligand antibodies can be synthesized from the nucleotide sequence by the method provided in Wells et al. in Int. J. Cancer 60, 137-144 (1995). The chimerized anti-EGFRML monoclonal antibody can be made in accordance with the methods described above. Humanized anti-EGFRML antibody can be prepared in accordance with the method described in example IV of PCT application WO 96/40210, which is incorporated herein by reference. Single chain anti-EGFRML antibodies (Fv anti-EGFRML) can be made in accordance with methods described by Wels et al. U.S. Pat. Nos. 6,129,915, 5,942,602 and 5,939,531 In addition to the biological molecules discussed above, the antagonists useful in the present invention may also be small molecules. Some examples of small molecules include organic compounds, organometallic compounds, salts of organic and organometallic compounds, saccharides, amino acids, and nucleotides. Small molecules shall further include molecules where their molecular weight is not greater than 600. Thus, small molecules may be lipids, oligosaccharides, oligopeptides, and oligonucleotides, and their derivatives, having a molecular weight of 600 or less. Pentamers of amino acids would be considered to be a small molecule.

It is emphasized that small molecules can have any molecular weight. They are merely called small molecules because they typically have molecular weights less than 600. Small molecules include compounds that are found in nature as well as synthetic compounds. Preferably, the small molecules inhibit the growth of refractory tumor cells that express EGFR/HER1. Pieczenik U.S. Pat. No. 5,866,363 describes methods of identifying and isolating binding peptides in the range of about 4 to about 12 amino acids and monoclonal and polyclonal antibodies with identifiable specificities which can act as an antagonist for any EGFR mitogenic ligand. It is hereby incorporated by reference.

Administration of EGFR/HER1 Mitogenic Ligand (EGFRML) Antagonists.

The present invention comprises administering an effective amount of the EGFRML antagonist to human patients. Administering the EGFRML antagonists can be accomplished in a variety of ways including systemically by the parenteral and enteral routes. For example, EGFRL antagonists of the present invention can easily be administered intravenously (e.g., intravenous injection) which is a preferred route of delivery. Intravenous administration can be accomplished by contacting the EGFRML antagonists with a suitable pharmaceutical carrier (vehicle) as understood by those skilled in the art. The EGFRLML antagonist may be administered with adjuvants, such as for example, BCG, immune system stimulators and chemotherapeutic agents.

EGFRML antagonists that are small molecule or biological drugs can be administered as described in Spada, U.S. Pat. No. 5,646,153 at column 57, line 47 to column 59, line 67. This patent is incorporated herein by reference.

The EGFRML ligand antagonists of the present invention are designed to inhibit the growth of refractory tumor cells when administered to a human patient in an effective amount. As used herein, an effective amount is that amount effective to achieve the specified result of inhibiting the growth of the refractory tumor.

Preferably, the EGFRML antagonist is provided to the tumor in an amount that inhibits tumor growth without disrupting the growth of normal tissue. Most preferably, the EGFRML antagonist inhibits tumor growth without serious side effects. Some serious side effects include bone marrow suppression, anemia and infection.

Optimal doses of EGFRML antagonists that are antibodies and functional equivalents of antibodies and AbMimetics can be determined by physicians based on a number of parameters including, for example, age, sex, weight, severity of the condition being treated, the antibody being administered, and the route of administration. For example, a concentration in excess of approximately 0.1 nM is normally sufficient.

As a rough guideline, in comparable clinical use of antibodies such as the “humanized” 3T6 antibodies (ReoPro TM), doses of antibodies in amounts of 10-300 mg/m.sup.2 weekly may be given. Equivalent doses of antibody fragments and peptides can be used at more frequent intervals.

Combination Therapy In one preferred embodiment the refractory tumor can be treated with an effective amount of an EGFRML antagonist with chemotherapeutic agents, radiation or combinations thereof.

Examples of chemotherapeutic agents or chemotherapy include alkylating agents, for example, nitrogen mustards, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; cytotoxic and cytostatic antibiotics and camptothecin derivatives.

Camptothecin derivatives include, for example camptothecin, 7-ethyl camptothecin, 10-hydroxy-7-ethyl-camptothecin (SN38), 9-amino camptothecin, 10,1-methylenedioxy-camptothecin (MDCPT) and topotecan. Such camptothecin derivatives also include lactone stable formulations of 7-ethyl-camptothecin disclosed in U.S. Pat. No. 5,604,233, the entire disclosure is incorporated herein by reference.

The present invention encompasses highly lipophilic camptothecin derivatives such as, for example, 10,11-methylenodioxy-camptothecin, 10,11-ethylenedioxy-camptothecin, 9-ethyl-camptothecin, 7-ethyl-10-hydroxy-camptothecin, 9-methyl-camptothecin, 9-chloro-10,11-methylenedioxy-camptothecin, 9-chloro camptothecin, 10-hydroxy-camptothecin, 9,10-dichloro camptothecin, 10-bromo-camptothecin, 10-chloro-camptothecin, 9-fluoro-camptothecin, 10-methyl-camptothecin, 10-fluoro-camptothecin, 9-methoxy-camptothecin, 9-chloro-7-ethyl-camptothecin and 11-fluoro-camptothecin. Such highly lipophilic camptothecin derivatives are disclosed in U.S. Pat. No. 5,880,133, the entire disclosure is incorporated herein by reference.

Water soluble camptothecin derivatives include, for example, the water soluble analog of camptothecin known as CPT-11,11-hydroxy-7-alkoxy-camptothecin, 11-hydroxy-7-methoxy camptothecin (11,7-HECPT) and 11-hydroxy-7-ethyl camptothecin (11,7-HECPT), 7-dimethylaminomethylene-10-,11-methylenedioxy-20 (R,S)-camptothecin, 7-dimethylaminomethylene-10,11-me-thylenedioxy-20(S)-camptothecin, 7-dimethylaminomethylene-10,11-ethylenedi-oxy-20(R,S)-camptothecin, and 7-morpholinomethylene-10,11-ethylenedioxy-20-(S)-camptothecin. Such water soluble camptothecin derivatives are disclosed in U.S. Pat. Nos. 5,559,235 and 5,468,754, the entire disclosures are incorporated herein by reference.

Preferred chemotherapeutic agents or chemotherapy include amifostine (ethyol), cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil, vinblastine, vincristine, bleomycin, paclitaxel (taxol), docetaxel (taxotere), aldesleukin, asparaginase, busulfan, carboplatin, cladribine, camptothecin, CPT-11,10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil and combinations thereof.

Administering chemotherapeutic agents can be accomplished in a variety of ways including systemically by the parenteral and enteral routes. Preferably, the chemotherapeutic agent is administered intravenously by contacting the chemotherapeutic agent with a suitable pharmaceutical carrier (vehicle) or excipient as understood by those skilled in the art. The dose of chemotherapeutic agent depends on numerous factors as is well known in the art. Such factors include age, sex, weight, severity of the condition being treated, the agent being administered, and the route of administration. For example, cisplatin may conveniently be administered at a dose of about 100 mg/m.sup.2. It should be emphasized, however, that the invention is not limited to any particular dose.

In yet another embodiment the refractory tumor can be treated with an effective amount of an EGFRML antagonist in combination with radiation. The source of radiation can be either external or internal to the patient being treated. When the source is external to the patient, the therapy is known as external beam radiation therapy (EBRT). When the source of radiation is internal to the patient, the treatment is called brachytherapy (BT).

The radiation is administered in accordance with well known standard techniques with standard equipment manufactured for this purpose, such as AECL Theratron and Varian Clinac. The dose of radiation depends on numerous factors as is well known in the art. Such factors include the organ being treated, the healthy organs in the path of the radiation that might inadvertently be adversely affected, the tolerance of the patient for radiation therapy, and the area of the body in need of treatment. The dose will typically be between 1 and 100 Gy, and more particularly between 2 and 80 Gy. The unit of dose is the gray (abbreviated Gy) which represents the absorption of an average of one joule of energy per kilogram of mass in the target material. It is equivalent to 100 rads. Some doses that have been reported include 35 Gy to the spinal cord, 15 Gy to the kidneys, 20 Gy to the liver, and 65-80 Gy to the prostate. It should be emphasized, however, that the invention is not limited to any particular dose. The dose will be determined by the treating physician in accordance with the particular factors in a given situation.

The distance between the source of the external radiation and the point of entry into the patient may be any distance that represents an acceptable balance between killing target cells and minimizing side effects. Typically, the source of the external radiation is between 70 and 100 cm from the point of entry into the patient.

Brachytherapy is generally carried out by placing the source of radiation in the patient. Typically, the source of radiation is placed approximately 0-3 cm from the tissue being treated. Known techniques include interstitial, intercavitary, and surface brachytherapy. The radioactive seeds can be implanted permanently or temporarily. Some typical radioactive atoms that have been used in permanent implants include iodine-125 and radon. Some typical radioactive atoms that have been used in temporary implants include radium, cesium-137, and iridium-192. Some additional radioactive atoms that have been used in brachytherapy include americium-241 and gold-198.

The dose of radiation for brachytherapy can be the same as that mentioned above for external beam radiation therapy. In addition to the factors mentioned above for determining the dose of external beam radiation therapy, the nature of the radioactive atom used is also taken into account in determining the dose of brachytherapy.

In the preferred embodiment, there is synergy when refractory tumors in human patients are treated with the EGFRML antagonist and chemotherapeutic agents or radiation or combinations thereof. In other words, the inhibition of tumor growth by the EGFRML antagonist is enhanced when combined with chemotherapeutic agents or radiation or combinations thereof. Synergy may be shown, for example, by greater inhibition of refractory tumor growth with combined treatment than would be expected from treatment with either the EGFRML antagonist, chemotherapeutic agent or radiation alone. Preferably, synergy is demonstrated by remission of the cancer where remission is not expected from treatment with EGFRML antagonist, chemotherapeutic agent or radiation alone.

The EGFRML antagonist is administered before, during, or after commencing chemotherapeutic agent or radiation therapy, as well as any combination thereof, i.e. before and during, before and after, during and after, or before, during, and after commencing the chemotherapeutic agent and/or radiation therapy. For example when the EGFRML antagonist is an antibody, it is typically administered between 1 and 30 days, preferably between 3 and 20 days, more preferably between 5 and 12 days before commencing radiation therapy and/or chemotherapeutic agents.

The combination treatment may also reduce the HAMA response to an mouse derived “humanized” antibody. It is known that both radiation and chemotherapy suppress and/or inhibit the immune system and its response to foreign antigens.

EXAMPLE 1 Clinical Trial In a clinical trial, human patients with refractory head and neck squamous cell carcinoma are treated with a combination of an EGRFML antagonist (chimeric anti-EGFRML monoclonal antibody) and cisplatin. The patients receive weekly infusions at loading/maintenance doses of 100/100, 400/250, or 500/250 mg/m.sup.2 in combination with 100 mg/m.sup.2 of cisplatin every three weeks.

EXAMPLE 2 Clinical Trial In a clinical trial, a human patient with refractory colon cancer is treated with a combination of an EGFR/HER1 ligand antagonist (chimeric anti-EGFRML monoclonal antibody) and CPT-11. The patient receives weekly infusions of C225 at a loading dose of 400 mg/m.sup.2 in combination with 125 mg/m.sup.2 of CPT-11. Maintenance doses of 250 mg/m.sup.2 C225 in combination with 69-125 mg/m.sup.2 of CPT-11 are administered on a weekly basis.

1. A method of inhibiting the growth of refractory tumors that are stimulated by a ligand of epidermal growth factor receptor (EGFR) in human patients, comprising treating the human patients with an effective amount of an EGFR/HER1 mitogenic ligand (EGFRML) antagonist.

2. A method according to claim 1 wherein the antagonist is a monoclonal antibody specific for EGFRML or a fragment that comprises the hypervariable region thereof.

3. A method according to claim 2 wherein the monoclonal antibody is chimerized and humanized.

4. A method according to claim 1 wherein the antagonist is a small molecule that binds specifically with EGFRML.

5. A method according to claim 1 wherein the refractory tumor has been treated with radiation or chemotherapy and combinations thereof.

6. A method according to claim 1 wherein the tumors are tumors of the breast, heart, lung, small intestine, colon, spleen, kidney, bladder, head and neck, ovary, prostate, brain, pancreas, skin, bone, bone marrow, blood, thymus, uterus, testicles, cervix, and liver.

7. A method of inhibiting the growth of refractory tumors that are stimulated by a ligand of epidermal growth factor receptor (EGFR) in human patients, comprising treating the human patients with an effective amount of a combination of EGFRML antagonist and radiation.

8. A method according to claim 8 wherein the antagonist is administered before, during and after radiation in all temporal combinations with null options included.

9. A method according to claim 8 wherein the source of the radiation is external to the human patient.

10. A method according to claim 8 wherein the source of radiation is internal to the human patient.

11. A method according to claim 8 wherein the antagonist is a monoclonal antibody.

12. A method according to claim 8 wherein the antagonist is a small molecule.

13. A method according to claim 8 wherein the tumors are tumors of the breast, heart, lung, small intestine, colon, spleen, kidney, bladder, head and neck, ovary, prostate, brain, pancreas, skin, bone, bone marrow, blood, thymus, uterus, testicles, cervix, and liver.

14. A method of inhibiting the growth of refractory tumors that are stimulated by a mitogenic ligand of epidermal growth factor receptor (EGFR) in human patients, comprising treating the human patients with an effective amount of an EGFRML antagonist and a chemotherapeutic agent.

15. A method according to claim 14 wherein the antagonist is administered before, during, and after treatment with the chemotherapeutic agent and all temporal combinations therein including no treatment times.

16. A method according to claim 14 wherein the chemotherapeutic agent is selected from the group consisting of amifostine, cisplatin, dacarbazine, dactinomycin, mechlorethamine, streptozocin, cyclophosphamide, carmustine, lomustine, doxorubicin, doxorubicin lipo, gemcitabine, daunorubicin, procarbazine, mitomycin, cytarabine, etoposide, methotrexate, 5-fluorouracil, vinblastine, vincristine, bleomycin, paclitaxel, docetaxel, aldesleukin, asparaginase, busulfan, carboplatin, cladribine, camptothecin, CPT-11,10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil and combinations thereof.

17. A method according to claim 14 wherein the chemotherapeutic agent is selected from the group consisting of cisplatin, doxorubicin, paclitaxel, CPT-11, topotecan and combinations thereof.

18. A method according to claim 14 wherein the tumors are tumors of the breast, heart, lung, small intestine, colon, spleen, kidney, bladder, head and neck, ovary, prostate, brain, pancreas, skin, bone, bone marrow, blood, thymus, uterus, testicles, cervix, and liver.

19. A method according to claim 14 wherein the antagonist is a monoclonal antibody.

20. A method according to claim 14 wherein the antagonist is a small molecule.
